(19) United States
(12) Patent Application Publication
(10) Pub. No.: US 2016/0272969 A1
(43) Pub. Date: Sep. 22, 2016

(54) COMPOSITIONS AND METHODS FOR MODULATING AUTOPHAGIC CELL DEATH

(71) Applicants: Ilana (Helena) NATHAN, Omer (IL); Abraham H. PAROLA, Omer (IL)

(72) Inventors: Ilana (Helena) NATHAN, Omer (IL); Abraham H. PAROLA, Omer (IL)

(21) Appl. No.: 15/033,389

(22) PCT Filed: Nov. 2, 2014
(86) PCT No.: PCT/IL2014/050949

§ 371 Date: Apr. 29, 2016

Related U.S. Application Data
(60) Provisional application No. 61/897,914, filed on Oct. 31, 2013.

(51) Int. Cl. C12N 15/113; A61K 31/18

(52) U.S. Cl. CPC C12N 15/113; A61K 31/18; C12N 2310/14; C12N 2320/30; C12N 2310/11

(57) ABSTRACT

The present invention relates to compositions and methods for modulating autophagic cell death, particularly by regulating alpha-1-antitrypsin activity, thereby useful for treating autophagy-associated diseases. In particular, the present invention relates to compositions and methods for treating diseases in which autophagy is impaired such as cancer and neurodegenerative diseases, as well as diseases in which autophagy is destructive (e.g., pancreatitis) as it is involved in unwanted cell death.

### FIELD OF THE INVENTION

The present invention relates to compositions and methods for modulating autophagic cell death, particularly by regulating alpha-1-antitrypsin activity, thereby useful for treating autophagy-associated diseases. In particular, the present invention relates to compositions and methods for treating diseases in which autophagy is impaired such as cancer and neurodegenerative diseases, as well as diseases in which autophagy is destructive (e.g., pancreatitis) as it is involved in unwanted cell death.

### BACKGROUND OF THE INVENTION

Autophagy is a catabolic process that mediates the turnover of intracellular constituents in a lysosome-dependent manner. Autophagy is initiated by the formation of an isolation membrane, which expands to engulf a portion of the cytoplasm to form a double membrane vesicle called the autophagosome. The autophagosome then fuses with a lysosome to form an autolysosome, where the captured material and the inner membrane are degraded by lysosomal hydrolases. Autophagy is therefore critical for the clearance of large protein complexes and defective organelles, and plays an important role in cellular growth, survival and homeostasis.

Autophagy has been primarily studied in unicellular eukaryotes, where it is known to be critical for survival of starvation conditions. When a unicellular eukaryote is cultured under conditions of nutrient deprivation, products of autophagic degradation, such as amino acids, fatty acids and nucleotides, can be used by the cell as structural components and as sources of energy. Cells in complex, multicellular eukaryotes, such as mammals, rarely experience nutrient deprivation under normal physiological conditions. However, when such cells undergo nutrient deprivation or cellular stress, autophagy is often upregulated, which enhances cell survival. Because of their rapid growth and genetic instability, cancer cells are more reliant on autophagy for survival and growth than untransformed cells (Ding et al., (2009), Mol. Cancer Ther., 8(7), 2036-2045).

Increasing number of studies propose autophagic cell death as the mechanism of action of some anticancer agents. These observations suggest that autophagic cell death induction in cancers may have a therapeutic value (Gozuacik and Kimchi, Oncogene (2004) 23, 2891-2906).

In addition to cancer and neurodegeneration, modulation of autophagy is a therapeutic strategy in a wide variety of additional diseases and disorders. For example, several liver diseases, cardiac diseases and muscle diseases are correlated with the accumulation of misfolded protein aggregates. In such diseases, agents that increase cellular autophagy may enhance the clearance of disease-causing aggregates and thereby contribute to treatment and reduce disease severity (Levine and Kroemer, (2008), Cell 132, 27-42). Additionally, elevated levels of autophagy have also been observed in pancreatic diseases, and have been demonstrated to be an early event in the progression of acute pancreatitis (Fortunato and Kroemer, (2009), Autophagy, 5(6)). Inhibitors of autophagy may, therefore, function as therapeutic agents in the treatment of pancreatitis.

Alpha-1 Antitrypsin

Alpha-1 Antitrypsin (AAT), also known as Alpha-1-Proteinase Inhibitor (API) and Serine Protease Inhibitor, is a plasma-derived protein belonging to the family of serine proteinase inhibitors. AAT is synthesized primarily in the liver, and to a lesser extent in other cells, including macrophages, intestinal epithelial cells and intestinal Paneth cells. In the liver, AAT is initially synthesized as a 52 kD precursor protein that subsequently undergoes post translational glycosylation at three asparagine residues, as well as tyrosine sulfonation. The resulting protein is secreted as a 55 kD native single-chain glycoprotein. AAT has a role in controlling tissue destruction by endogenous serine proteinases, and is the most prevalent serine proteinase inhibitor in blood plasma. AAT inhibits, inter alia, trypsin, chymotrypsin, various types of elastases, skin collagenase, renin, urokinase and proteases of polymorphonuclear lymphocytes.

AAT has anti-inflammatory properties, providing protection from tissue damage in the kidney, lung and liver. International Patent Application No. WO 92/06706 provides use of an effective amount of alpha 1-antitrypsin among other serine protease inhibitors for the prophylaxis or treatment of a mast cell-implicated disease or injury in a mammal. U.S. Patent Application No. 2008/0095806 provides compositions comprising a protease inhibitor, inter alia, alpha 1-antitrypsin, useful for preventing and treating hyperproliferative and inflammatory mucocutaneous disorders. U.S. Pat. No. 5,134,119 discloses a method for prophylaxis or direct treatment of mast cell implicated skin inflammation or treating the symptoms of burns in a patient comprising administering an effective amount of an analog of alpha 1-antitrypsin. U.S. Pat. No. 5,093,316 discloses a method and pharmaceutical compositions for treating pulmonary inflammation in pulmonary diseases comprising administering an effective amount of microcrystalline alpha-1-antitrypsin, derivatives or salts thereof.

U.S. Pat. No. 7,419,670 discloses viral protein SERP-1, SERP-1 analogs or biologically active fragments, which are useful for treating inflammatory or immune reaction associated with arthritis, systemic lupus erythematosus (SLE), multiple sclerosis (MS) and asthma. While U.S. Pat. No. 7,419,670 claims methods of treating a mammalian subject having arthritis, systemic lupus erythematosus (SLE), multiple sclerosis (MS) and asthma the peptides disclosed are useful only when administered in combination with an immunosuppressant.

U.S. Patent Application No. 2008/0261868 provides a method of treating a subject suffering from a disease characterized by excessive apoptosis by administering at least one serine protease inhibitor, preferably alpha 1-antitrypsin or a derivative thereof.

International Patent Application No. WO 10/029537, by an inventor of the present invention and coworkers, relates to the use of alpha-1-antitrypsin as an anti-necrotic agent, and provides methods for the treatment of tissue necrosis by administration of alpha-1-antitrypsin.

None of the background art, however, discloses or suggests that modulating AAT activity regulates autophagic cell death, thereby useful in treating pathologies associated with impaired or harmful autophagy.

There exists a long-felt need effective means of modulating autophagy in cells thereby curing or ameliorating autophagy-associated diseases, including but not limited to, cancer, neurodegenerative diseases and pancreatitis.

### SUMMARY OF THE INVENTION

The present invention provides compositions and methods for regulating alpha-1-antitrypsin (AAT) mediated autophagic activity, thereby treating autophagy-associated pathologies. In particular, the present invention relates to compositions and methods for treating diseases associated with impaired autophagy including but not limited to cancer and neurodegenerative diseases, as well as treating diseases and disorders associated with unwanted autophagy (e.g., pancreatitis).

It is now disclosed for the first time that AAT is a regulator of autophagic cell death. Surprisingly, inhibition or reduction of AAT activity induces autophagic cell death in breast and colon cancer cells. Further, treating cells with AAT lead to remarkable inhibition of autophagic cell death.

Thus, in some embodiments, the present invention provides compositions and methods for treating a disease or disorder associated with aberrant autophagic activity. In another embodiment, the present invention provides compositions and methods for inducing autophagic cell death thereby treating a hyperproliferative disease, including but not limited to cancer or a premalignant tumors. In yet another embodiment, the present invention provides compositions and methods for reducing autophagic cell death thereby treating a disease or disorder characterized by excessive autophagic cellular death, including but not limited to neurodegenerative diseases.

According to some embodiment of the present invention, autophagic cell death is mediated by regulating alpha-1-antitrypsin expression or activity in said cell. According to particular embodiments of the invention, the alpha-1-antitrypsin is a mammal or a human alpha-1-antitrypsin. According to another embodiment, the human alpha-1-antitrypsin comprises the amino acid sequence as set forth in SEQ ID NO: 1, or a variant, an active analog or fragment thereof. According to another embodiment, the human alpha-1-antitrypsin consists of the amino acid sequence as set forth in SEQ ID NO: 1 (GenBank accession No. ABV21360.1). According to another particular embodiment, the human alpha-1-antitrypsin is encoded by polynucleotide having a nucleic acid sequence as set forth in SEQ ID NO: 2, or an active analog or fragment thereof. According to yet another particular embodiment, the human alpha-1-antitrypsin is encoded by polynucleotide (mRNA) having of a nucleic acid sequence selected from the group consisting of: accession No. NM_000295.4, NM_001002236.2, NM_001002235.2, NM_001127700.1, NM_001127701.1, NM_001127702.1, NM_001127703.1, NM_001127704.1, NM_001127705.1, NM_001127706.1 or NM_001127707.1. Each possibility represents a separate embodiment of the invention. According to yet another particular embodiment, the human alpha-1-antitrypsin is encoded by polynucleotide consisting of a nucleic acid sequence as set forth in SEQ ID NO: 2 (accession No. NM_000295.4).

According to a first aspect, the present invention provides a method for treating cancer in a subject in need thereof, comprising administering to the subject a therapeutically effective amount of an agent that reduces alpha-1-antitrypsin activity or expression levels, thereby treating cancer in said subject.

According to another embodiment, the agent is a hybridizing agent capable of hybridizing to nucleic acid encoding alpha-1-antitrypsin. According to another embodiment, the hybridizing agent comprises at least one nucleic acid sequence at least 85% complementary to a target sequence of about 12 to about 100 nucleotides of alpha-1-antitrypsin mRNA. According to another embodiment, the target sequence is from about 12 to about 50 nucleotides of alpha-1-antitrypsin mRNA. According to yet another embodiment, said target sequence is from about 12 to about 25 nucleotides of alpha-1-antitrypsin mRNA.

According to another embodiment, the hybridizing agent is selected from an RNA interference (RNAi) molecule and an antisense molecule.

According to additional embodiments, the hybridizing agent is an RNAi molecule selected from a short interference RNA (siRNA), small hairpin RNA (shRNA) and microRNA (miRNA). Each possibility represents a separate embodiment of the invention. According to another embodiment, the RNAi molecule comprises: (a) a first polynucleotide having at least 90% identity to the target sequence of alpha-1-antitrypsin mRNA; and (b) a second polynucleotide sequence substantially complementary to the first polynucleotide; wherein the first and the second polynucleotide sequences are annealed to each other to form the RNAi molecule.

According to another embodiment, the hybridizing agent is an antisense molecule comprising a polynucleotide at least 90% complementary to a target sequence of alpha-1-antitrypsin.

According to another embodiment, the agent is a serine protease inhibitor. In an exepmlary embodiment, the serine protease inhibitor is N-tosyl-L-phenylalanine chloromethyl ketone (TPCK).

According to another embodiment, the agent is administered to said subject in the form of a pharmaceutical composition further comprising a pharmaceutically acceptable carrier, excipient or diluent.

According to a specific embodiment, the cancer is a hematopoietic malignancy. According to another specific embodiment, the hematopoietic malignancy is selected from the group consisting of: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma and non-Hodgkin's lymphoma. Each possibility represents a separate embodiment of the invention.

According to a specific embodiment, the cancer is a solid malignancy. According to another specific embodiment, the solid malignancy is selected from the group consisting of: prostate cancer, breast cancer, skin cancer, colon cancer, lung cancer, pancreatic cancer, head and neck cancer, kidney cancer, ovarian cancer, cervix cancer, bone cancer, liver cancer, thyroid cancer and brain cancer. Each possibility represents a separate embodiment of the invention.

In a particular embodiment, the solid malignancy is breast cancer. In another particular embodiment, the solid malignancy is colon cancer. In yet another particular embodiment, the colon cancer is colon adenocarcinoma.

According to some embodiments, the subject is a mammal. According to a particular embodiment, the subject is a human.

According to another aspect, the present invention provides a method for treating a disease or disorder associated with excessive autophagy in a subject in need thereof, comprising administering to the subject a therapeutically effective amount of an active agent selected from (a) an isolated alpha-1-antitrypsin polypeptide comprising the amino acid sequence as set forth in SEQ ID NO: 1, or an active analog or fragment thereof; (b) an isolated nucleic acid molecule encoding alpha-1-antitrypsin polypeptide, the alpha-1-antitrypsin polypeptide comprises the amino acid sequence as set forth in SEQ ID NO: 1, or an active analog or fragment thereof; or (c) an expression vector comprising the isolated nucleic acid molecule of (b); thereby treating the disease or disorder associated with excessive autophagy in said subject. Each possibility represents a separate embodiment of the invention.

According to another embodiment, the nucleic acid molecule encoding alpha-1-antitrypsin has a nucleic acid sequence as set forth in SEQ ID NO: 2 or an active analog thereof.

According to another embodiment, the alpha-1-antitrypsin is administered to said subject in the form of a pharmaceutical composition further comprising a pharmaceutically acceptable carrier, excipient or diluent.

According to another embodiment, the disease or disorder associated with excessive autophagy is a neurodegenerative disease. According to some embodiments, the neurodegenerative disease is selected from the group consisting of: Alzheimer's disease, Huntington's disease, Parkinson's disease, neurodegeneration due to stroke, amyotrophic lateral sclerosis (ALS), prion disease, Pick's disease, Progressive Supranuclear Palsy (PSP), fronto-temporal dementia (FTD), pallido-ponto-nigral degeneration (PPND), Guam-ALS syndrome, pallido-nigro-luysian degeneration (PNLD) and cortico-basal degeneration (CBD). Each possibility represents a separate embodiment of the invention.

According to a particular embodiment, said disease or disorder is associated with cell death. According to another particular embodiment, said disease or disorder is associated with neuronal cell death. According to another embodiment, the disease or disorder associated with excessive autophagy is pancreatitis. According to another embodiment, the disease or disorder associated with excessive autophagy is a myopathy disease including but not limited to Danon disease, X-linked myopathy with excessive autophagy, infantile autophagic vacuolar myopathy, adult-onset vacuolar myopathy with multiorgan involvement, X-linked congenital autophagic vacuolar myopathy. According to another embodiment the present invention provides a method for treating a disease or disorder associated with excessive autophagy in a subject in need thereof, comprising administering to the subject a therapeutically effective amount of an active agent capable of modulating alpha-1-antitrypsin (AAT) expression levels or activity, thereby treating the disease or disorder associated with excessive autophagy in said subject. In one embodiment, said agent up-regulates AAT expression. In another embodiment, said agent decreases AAT degradation.

According to another aspect, the present invention provides a method of screening for an autophagic modulating agent, comprising identifying agents, which modulate AAT activity or expression.

According to some embodiments, the method comprises:

(a) exposing a cell expressing AAT to a putative autophagic modulating agent;

(b) determining the expression or activity of ATT;

wherein a difference of AAT expression or activity indicates that the agent is an autophagic modulating agent.

In one embodiment, reduction (or inhibition) of AAT expression or activity indicates that the agent is an autophagic inducing agent. In a specific embodiment, the autophagic inducing agent is useful in treating a hyperproliferative disease such as cancer.

According to another embodiment, the method further comprises step (c) determining the change in survival or autophagic death of the cell in the presence of the agent relative to a control.

According to specific embodiments, the putative autophagic modulating agent is selected from the group consisting of: peptides, nucleic acids, organic molecules, inorganic compounds and antibodies or antigen binding fragments thereof.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows that TPCK induced autophagy as assessed by different methods. Autophagy induction is evident by fluorescence microscopy in MCF-7 (A) and HT-29 (C) cells treated with TPCK, with or without 3 MA (5 mM) or bafilomycin A1 (50 nM) for 24 hours, and stained with MDC; magnification ×40. The quantification of the results obtained with MCF-7 (B) and HT-29 (D) cells. Images of GFP-LC3 transfected MCF-7 cells treated with TPCK (50 μM) for 24 hours (E). MCF-7 (F, G) and HT-29 (H, I) cells treated with TPCK (50 μM) for different times, and LC3-II levels were analyzed by western blots. *P<0.05, **P<0.01, #P<0.001, compared with control, by Student t test.

FIG. 2 presents results demonstrating interaction between alpha-1-antitrypsin (AAT) and (N-tosyl-L-phenylalanine chloromethyl ketone) TPCK, a chymotrypsin-like protease inhibitor. (A) SDS-PAGE electrophoresis and fluorescence detection of a fluorescent analog of TPCK, TRFCK (track 1), and Western blot analysis of AAT (track

FIG. 3 demonstrates AAT role in autophagic cell death inhibition in MCF-7 cells assessed by monodansylcadaverine (MDC) (A) or by trypan blue (B) staining.

FIG. 4 presents results indicating that TPCK inhibits AAT inhibition of trypsin as measured by enzymatic assay.

FIG. 5 demonstrates that AAT modulates autophagy. (a) It is shown that intracellular levels of AAT are downregulated during autophagy induced by TPCK (50 μM) and tamoxifen (10 μM) in MCF-7 (A, B) and HT-29 (C, D) cells, detected by immunobloting. (b) It is demonstrated that AAT silencing causes autophagy induction. MCF-7 cells were transfected with siRNAs, and LC3-II and AAT levels analyzed by immunobloting (E, F). (c) It is also shown that AAT inhibits autophagic cell death induced by TPCK (50 μM, 24 h) or tamoxifen (5 μM, 72 h) in MCF-7 cells as seen (G) and analyzed (F) by fluorescence microscopy and measured by trypan blue staining (I). magnification ×40. Data represent mean±SEM of at least three independent experiments. *P<0.05, **P<0.01, ***P<0.001 (versus control); #P<0.05; ##P<0.01 (versus Without AAT), by the Student t test.

FIG. 6 shows the amino acid (SEQ ID NO: 1) and nucleic acid (SEQ ID NO: 2) sequence

### DETAILED DESCRIPTION OF THE INVENTION

The present invention provides compositions and methods for regulating alpha-1-antitrypsin (AAT) mediated autophagic activity, thereby treating pathologies associated with impaired or deleterious autophagy. In particular, the present invention relates to compositions and methods for treating cancer and neurodegenerative diseases.

As exemplified herein below AAT plays a role in mediating autophagic cell death. Reduction of AAT protein levels unexpectedly resulted in autophagic cell death of MCF-7 breast cancer cells and HT-29 human colon adenocarcinoma cells (Example 5). Furthermore, addition of AAT to TPCK and tamoxifen (autophagic cell death inducers) treated cells, inhibited autophagic cell death (Example 5).

Thus, in some embodiments, the present invention provides methods for treating a disease or disorder associated with aberrant autophagic activity. In another embodiment, the present invention provides methods for modulating autophagic cell death. In another embodiment, the present invention provides methods for inducing autophagic cell death thereby treating a hyperproliferative disease, including but not limited to cancer or a premalignant tumors. In yet another embodiment, the present invention provides methods for reducing autophagic cell death thereby treating a disease or disorder characterized by excessive cellular death, including but not limited to a neurodegenerative disease.

The term “modulating autophagic cell death” as used herein refers to the activation (e.g., enhancement) or reduction (e.g., inhibition) of alpha-1 antitrypsin (AAT) activity or expression. Modulating AAT autophagic activity, in some embodiment, relates to promoting or inducing cell autophagy. In some embodiments, modulating AAT autophagic activity relates to inhibiting or reducing cell autophagy. Methods for assaying cell autophagy are well known in the art and include monodansylcadaverine (MDC) staining for in vivo labeling of autophagic vacuoles, antibodies specific to LC3 to identify LC3-positive structures such as autophagosomes, Cyto-ID™ Autophagy detection kit (Enzo Life sciences, Inc.). Further assays for monitoring and assaying cell autophagy are reviewed in Klionsky et al. Autophagy 3:3, 181-206; May/June 2007.

The term “autophagy associated disease” includes a disease that can be treated by the autophagy modulation. Examples of such diseases include diseases caused by misfolded protein aggregates. The term “disease caused by misfolded protein aggregates” is intended to include any disease, disorder or condition associated with or caused by misfolded protein aggregates. For example, such diseases include Alzheimer's disease, Parkinson's disease, amyotrophic lateral sclerosis, Huntington's disease, spinocerebellar ataxia, oculopharyngeal muscular dystrophy, prion diseases, fatal familial insomnia, alpha-1 antitrypsin deficiency, dentatorubral pallidoluysian atrophy, frontal temporal dementia, progressive supranuclear palsy, x-linked spinobulbar muscular atrophy, and neuronal intranuclear hyaline inclusion disease. The term “autophagy associated disease” also includes cancer e.g., any cancer wherein the induction of autophagy would inhibit cell growth and division, reduce mutagenesis, remove mitochondria and other organelles damaged by reactive oxygen species or kill developing tumor cells. Autophagy associated diseases can be chronic diseases.

In another embodiment, reduction of alpha-1-antitrypsin (AAT) activity or expression level is at least a 10% reduction as compared to the control (e.g., without an AAT activity-reducing agent). Thus, the reduction can be a 10, 20, 30, 40, 50, 60, 70, 80, 90, 100% or any amount of reduction in between the specifically recited percentages, as compared to native or control levels. In some embodiments, the AAT activity-reducing agent induces auothophagic cell death, of at least 90%, 85%, 80%, 75%, 70%, 60%, 50%, 40%, 30%, 20%, 10% in a population of cells in which the AAT activity-reducing agent is present than compared to a control cell population where the agent is not present.

In another embodiment, enhancing (or promoting) AAT activity or expression levels is at least a 10% elevation of AAT activity or expression as compared to the native or control levels. Thus, the elevation can be a 10, 20, 30, 40, 50, 60, 70, 80, 90, 100%, or any amount of elevation in between the specifically recited percentages, as compared to native or control levels. In some embodiments, the elevation of AAT activity reduces auothophagic cell death, of at least 90%, 85%, 80%, 75%, 70%, 60%, 50%, 40%, 30%, 20%, 10% in a population of cells compared to a control cell population.

In some embodiment, the present invention provides methods of AAT gene silencing for treatment of malignant disease. The term “gene silencing” as used herein refers to a process by which the expression of a specific gene product is lessened or attenuated. Gene silencing can take place by a variety of pathways. Unless specified otherwise, as used herein, gene silencing refers to decreases in gene product expression. Gene silencing may result from RNA interference (RNAi), a defined, though partially characterized pathway whereby short interference RNA (siRNA) act in concert with host proteins (e.g., the RNA induced silencing complex, RISC) to degrade messenger RNA (mRNA) in a sequence-dependent fashion. The term “RNAi molecule” or “RNAi polynucleotide” refers to single- or double-stranded RNA molecules typically having a total of from about 15 to about 100 bases, preferably from about 20 to about 60 bases and comprises both a sense and antisense sequence. For example, the RNA interference molecule can be a double-stranded polynucleotide molecule comprising self-complementary sense and antisense regions, wherein the antisense region comprises complementarity to a target nucleic acid molecule. Alternatively the RNAi molecule can be a single-stranded hairpin polynucleotide having self-complementary sense and antisense regions, wherein the antisense region comprises complementarity to a target nucleic acid molecule or it can be a circular single-stranded polynucleotide having two or more loop structures and a stem comprising self-complementary sense and antisense regions, wherein the antisense region comprises complementarity to a target nucleic acid molecule, and wherein the circular polynucleotide can be processed either in vivo or in vitro to generate an active molecule capable of mediating RNAi.

The level of gene silencing can be measured by a variety of means, including, but not limited to, measurement of transcript levels by Northern Blot Analysis, Branched-DNA techniques, transcription-sensitive reporter constructs, expression profiling (e.g., DNA chips), and related technologies. Alternatively, the level of silencing can be measured by assessing the level of the protein encoded by a specific gene/polynucleotide. This can be accomplished by performing a number of studies including Western Analysis, measuring the levels of expression of a reporter protein that has e.g., fluorescent properties (e.g., GFP) or enzymatic activity (e.g., alkaline phosphatases), or several other procedures.

As used herein, the terms “target”, “target sequence” or “target gene” refer to the nucleic acid sequence that is selected for silencing. The target sequence can be RNA or DNA, and may also refer to a polynucleotide comprising the target sequence.

The term “gene” refers to a nucleic acid (e.g., DNA or RNA) sequence that comprises coding sequences necessary for the production of RNA or a polypeptide. A polypeptide can be encoded by a full-length coding sequence or by any part thereof. The fragments may range in size from a few nucleotides to the entire gene sequence minus one nucleotide.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated (or untranslated) sequences (5′ UTR). The sequences which are located 3′ or downstream of the coding region and which are present on the mRNA are referred to as 3′ non-translated (or untranslated) sequences (3′ UTR).

As used herein, the term “nucleotide” refers to a ribonucleotide or a deoxyribonucleotide or modified form thereof, as well as an analog thereof. Nucleotides include species that comprise purines, e.g., adenine, hypoxanthine, guanine, and their derivatives and analogs, as well as pyrimidines, e.g., cytosine, uracil, thymine, and their derivatives and analogs. The term nucleotide also includes those species that have a detectable label, such as for example a radioactive or fluorescent moiety, or mass label attached to the nucleotide.

Nucleotide analogs include nucleotides having modifications in the chemical structure of the base, sugar and/or phosphate, including, but not limited to, 5-position pyrimidine modifications, 8-position purine modifications, modifications at cytosine exocyclic amines, and substitution of 5-bromo-uracil; and 2′-position sugar modifications, including but not limited to, sugar-modified ribonucleotides in which the 2′-OH is replaced by a group such as an H, OR, R, halo, SH, SR, NH₂, NHR, NR₂, or CN, wherein R is an alkyl moiety. Nucleotide analogs are also meant to include nucleotides with bases such as inosine, queuosine, xanthine, sugars such as 2′-methyl ribose, non-natural phosphodiester linkages such as methylphosphonates, phosphorothioates and peptides.

The term “polynucleotide” refers to polymers of nucleotides, and includes but is not limited to DNA, RNA, DNA/RNA hybrids including polynucleotide chains of regularly and/or irregularly alternating deoxyribosyl moieties and ribosyl moieties (i.e., wherein alternate nucleotide units have an —OH, then an —H, then an —OH, then an —H, and so on at the 2′ position of a sugar moiety), and modifications of these kinds of polynucleotides, wherein the attachment of various entities or moieties to the nucleotide units at any position are included.

“Complementarity” as used herein refers to the ability of a nucleic acid to form hydrogen bond(s) with another nucleic acid sequence. A percent complementarity indicates the percentage of contiguous residues in a nucleic acid molecule, which can form hydrogen bonds (e.g., Watson-Crick base pairing) with a second nucleic acid sequence. “Fully complementary” means that all the contiguous residues of a nucleic acid sequence will hydrogen bond with the same number of contiguous residues in a second nucleic acid sequence. The term “substantially” complementary as used herein refers to a molecule in which about 80% of the contiguous residues of a nucleic acid sequence will hydrogen bond with the same number of contiguous residues in a second nucleic acid sequence. In some embodiments substantially complementary refers to 85%, 90%, 95% of the contiguous residues of nucleic acid sequence hydrogen bonding with the same number of contiguous residues in a second nucleic acid sequence.

The term “homology”, as used herein, refers to a degree of sequence similarity in terms of shared amino acid or nucleotide sequences. There may be partial homology or complete homology (i.e., identity). As used herein, the term “at least” with regard to a certain degree of homology encompasses any degree of homology from the specified percentage up to 100%.

For amino acid sequence homology, amino acid similarity matrices may be used as are known in different bioinformatics programs (e.g. BLAST, Smith Waterman). Different results may be obtained when performing a particular search with a different matrix. Homologous peptide or polypeptides are characterized by one or more amino acid substitutions, insertions or deletions, such as, but not limited to, conservative substitutions, provided that these changes do not affect the biological activity of the peptide or polypeptide as described herein.

Degrees of homology for nucleotide sequences are based upon identity matches with penalties made for gaps or insertions required to optimize the alignment, as is well known in the art (e.g. Altschul S F et al., 1990; J MoI Biol 215(3), 403-10; Altschul S F et al., 1997; Nucleic Acids Res 25, 3389-3402). The degree of sequence homology is presented in terms of percentage, e.g. “70% homology”.

The term “construct” as used herein refers to an artificially assembled or isolated nucleic acid molecule, which includes the polynucleotide/gene of interest. In general, a construct may include the polynucleotide(s) of interest, a marker gene that in some cases can also be the gene of interest and appropriate regulatory sequences. It should be appreciated that the inclusion of regulatory sequences in a construct is optional, for example, such sequences may not be required in situations where the regulatory sequences of a host cell are to be used. The term construct includes vectors but should not be seen as being limited thereto.

The term “expression vector” and “recombinant expression vector” as used herein refers to a DNA molecule, for example a plasmid or virus, containing a desired and appropriate nucleic acid sequences necessary for the expression of the operably linked polynucleotide of interest in a particular host cell. The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the invention can be operably linked, either directly or indirectly, 5′ to the target mRNA, or 3′ to the target mRNA, or within the target mRNA, or a first complementary region is 5′ and its complement is 3′ to the target mRNA.

The terms “promoter element,” “promoter,” or “promoter sequence” as used herein, refer to a DNA sequence that is located at the 5′ end (i.e. precedes) the protein coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene/polynucleotide. The promoter, therefore, serves as a transcriptional regulatory element and also provides a site for initiation of transcription of the gene into mRNA. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity. Promoters which cause a gene to be expressed in most cell types at most times are commonly referred to as “constitutive promoters”. Promoters that derive gene expression in a specific tissue are called “tissue specific promoters”. Tissue specific promoters can be expressed constitutively or their expression may require a specific induction.

As used herein, the term an “enhancer” refers to a DNA sequence, which can stimulate promoter activity, and may be an innate element of the promoter or a heterologous element inserted to enhance the level or tissue-specificity of a promoter. The term “expression”, as used herein, refers to the production of a functional end-product e.g., an mRNA or a protein.

The terms “polypeptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

The term “isolated peptide” refers to a peptide that is essentially free from contaminating cellular components, such as carbohydrate, lipid, or other proteinaceous impurities associated with the peptide in nature. Typically, a preparation of isolated peptide contains the peptide in a highly purified form, i.e., at least about 80% pure, at least about 90% pure, at least about 95% pure, greater than 95% pure, or greater than 99% pure.

One of skill in the art will recognize that individual substitutions, deletions or additions to a peptide, or protein sequence which alters, adds or deletes a single amino acid or a small percentage of amino acids in the encoded sequence is a conservatively modified variant where the alteration results in the substitution of an amino acid with a similar charge, size, and/or hydrophobicity characteristics, such as, for example, substitution of a glutamic acid (E) to aspartic acid (D). Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W) (see, e.g., Creighton, Proteins, 1984).

The term “analog” includes any peptide having an amino acid sequence substantially identical to one of the sequences specifically shown herein in which one or more residues have been conservatively substituted with a functionally similar residue and which displays the abilities as described herein. Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another. Each possibility represents a separate embodiment of the present invention.

The phrase “conservative substitution” also includes the use of a chemically derivatized residue in place of a non-derivatized residue provided that such peptide displays the requisite function of as specified herein.

Typically, the present invention encompasses derivatives of the polypeptides. The term “derivative” or “chemical derivative” includes any chemical derivative of the polypeptide having one or more residues chemically derivatized by reaction of side chains or functional groups. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those peptides, which contain one or more naturally occurring amino acid derivatives of the twenty standard amino acid residues. For example: 4-hydroxyproline may be substituted for proline; 5-hydroxylysine may be substituted for lysine; 3-methylhistidine may be substituted for histidine; homoserine may be substituted or serine; and ornithine may be substituted for lysine.

In addition, a polypeptide derivative can differ from the natural sequence of the peptides of the invention by chemical modifications including, but are not limited to, terminal-NH₂ acylation, acetylation, or thioglycolic acid amidation, and by terminal-carboxlyamidation, e.g., with ammonia, methylamine, and the like. Polypeptides can be either linear, cyclic or branched and the like, which conformations can be achieved using methods well known in the art.

The peptide or polypeptides derivatives and analogs according to the principles of the present invention can also include side chain bond modifications, including but not limited to —CH₂—NH—, —CH₂—S—, —CH₂—S═O, O═C—NH—, —CH₂—O—, —CH₂—CH₂—, S═C—NH—, and —CH═CH—, and backbone modifications such as modified peptide bonds. Peptide bonds (—CO—NH—) within the peptide can be substituted, for example, by N-methylated bonds (—N(CH3)-CO—); ester bonds (—C(R)H—C—O—O—C(R)H—N); ketomethylene bonds (—CO—CH2-); α-aza bonds (—NH—N(R)—CO—), wherein R is any alkyl group, e.g., methyl; carba bonds (—CH2-NH—); hydroxyethylene bonds (—CH(OH)—CH2-); thioamide bonds (—CS—NH); olefinic double bonds (—CH═CH—); and peptide derivatives (—N(R)—CH2-CO—), wherein R is the “normal” side chain, naturally presented on the carbon atom. These modifications can occur at one or more of the bonds along the peptide chain and even at several (e.g., 2-3) at the same time.

The present invention also encompasses peptide or polypeptides derivatives and analogs in which free amino groups have been derivatized to form amine hydrochlorides, p-toluene sulfonylamino groups, carbobenzoxyamino groups, t-butyloxycarbonylamino groups, chloroacetylamino groups or formylamino groups. Free carboxyl groups may be derivatized to form, for example, salts, methyl and ethyl esters or other types of esters or hydrazides. The imidazole nitrogen of histidine can be derivatized to form N-im-benzylhistidine.

The peptide analogs can also contain non-natural amino acids. Examples of non-natural amino acids include, but are not limited to, sarcosine (Sar), norleucine, ornithine, citrulline, diaminobutyric acid, homoserine, isopropyl Lys, 3-(2′-naphtyl)-Ala, nicotinyl Lys, amino isobutyric acid, and 3-(3′-pyridyl-Ala).

Furthermore, the peptide or polypeptides analogs can contain other derivatized amino acid residues including, but not limited to, methylated amino acids, N-benzylated amino acids, O-benzylated amino acids, N-acetylated amino acids, O-acetylated amino acids, carbobenzoxy-substituted amino acids and the like. Specific examples include, but are not limited to, methyl-Ala (MeAla), MeTyr, MeArg, MeGlu, MeVal, MeHis, N-acetyl-Lys, O-acetyl-Lys, carbobenzoxy-Lys, Tyr-O-Benzyl, Glu-O-Benzyl, Benzyl-His, Arg-Tosyl, t-butylglycine, t-butylalanine, phenylglycine, cyclohexylalanine, and the like.

The peptides or polypeptides of the invention may be synthesized or prepared by techniques well known in the art. The peptides can be synthesized by a solid phase peptide synthesis method of Merrifield (see J. Am. Chem. Soc., 85:2149, 1964). Alternatively, the peptides of the present invention can be synthesized using standard solution methods well known in the art (see, for example, Bodanszky, M., Principles of Peptide Synthesis, Springer-Verlag, 1984) or by any other method known in the art for peptide synthesis.

In general, these methods comprise sequential addition of one or more amino acids or suitably protected amino acids to a growing peptide chain bound to a suitable resin.

Normally, either the amino or the carboxyl group of the first amino acid is protected by a suitable protecting group. The protected or derivatized amino acid can then be either attached to an inert solid support (resin) or utilized in solution by adding the next amino acid in the sequence having the complimentary (amino or carboxyl) group suitably protected, under conditions conductive for forming the amide linkage. The protecting group is then removed from this newly added amino acid residue and the next amino acid (suitably protected) is added, and so forth. After all the desired amino acids have been linked in the proper sequence, any remaining protecting groups are removed sequentially or concurrently, and the peptide chain, if synthesized by the solid phase method, is cleaved from the solid support to afford the final peptide.

In the solid phase peptide synthesis method, the alpha-amino group of the amino acid is protected by an acid or base sensitive group. Such protecting groups should have the properties of being stable to the conditions of peptide linkage formation, while being readily removable without destruction of the growing peptide chain. Suitable protecting groups are t-butyloxycarbonyl (BOC), benzyloxycarbonyl (Cbz), biphenylisopropyloxycarbonyl, t-amyloxycarbonyl, isobornyloxycarbonyl, (alpha,alpha)-dimethyl-3,5dimethoxybenzyloxycarbonyl, o-nitrophenylsulfenyl, 2-cyano-t-butyloxycarbonyl, 9-fluorenylmethyloxycarbonyl (FMOC) and the like. The BOC or FMOC protecting group is preferred.

In the solid phase peptide synthesis method, the C-terminal amino acid is attached to a suitable solid support. Suitable solid supports useful for the above synthesis are those materials, which are inert to the reagents and reaction conditions of the stepwise condensation-deprotection reactions, as well as being insoluble in the solvent media used. Suitable solid supports are chloromethylpolystyrene-divinylbenzene polymer, hydroxymethyl-polystyrene-divinylbenzene polymer, and the like. The coupling reaction is accomplished in a solvent such as ethanol, acetonitrile, N,N-dimethylformamide (DMF), and the like. The coupling of successive protected amino acids can be carried out in an automatic polypeptide synthesizer as is well known in the art.

The polypeptides of the invention may alternatively be synthesized such that one or more of the bonds, which link the amino acid residues of the peptides are non-peptide bonds. These alternative non-peptide bonds include, but are not limited to, imino, ester, hydrazide, semicarbazide, and azo bonds, which can be formed by reactions well known to skilled in the art.

The polypeptides of the present invention, analogs or derivatives thereof produced by recombinant techniques can be purified so that the peptides will be substantially pure when administered to a subject. The term “substantially pure” refers to a compound, e.g., a peptide, which has been separated from components, which naturally accompany it. Typically, a peptide is substantially pure when at least 50%, preferably at least 75%, more preferably at least 90% and most preferably at least 99% of the total material (by volume, by wet or dry weight, or by mole percent or mole fraction) in a sample is the peptide of interest. Purity can be measured by any appropriate method, e.g., in the case of peptides by HPLC analysis.

Included within the scope of the invention are polypeptide conjugates comprising the peptides of the present invention derivatives, or analogs thereof joined at their amino or carboxy-terminus or at one of the side chains via a peptide bond to an amino acid sequence of a different protein. Conjugates comprising peptides of the invention and a protein can be made by protein synthesis, e. g., by use of a peptide synthesizer, or by ligating the appropriate nucleic acid sequences encoding the desired amino acid sequences to each other by methods known in the art, in the proper coding frame, and expressing the conjugate by methods commonly known in the art. Additionally or alternatively, the polypeptides of the present invention, derivatives, or analogs thereof can be joined to another moiety such as, for example, a fatty acid, a sugar moiety, arginine residues, hydrophobic moieties, and any known moiety that facilitate membrane or cell penetration.

Addition of amino acid residues may be performed at either terminus of the peptides of the invention for the purpose of providing a “linker” by which the peptides of this invention can be conveniently bound to a carrier. Such linkers are usually of at least one amino acid residue and can be of 40 or more residues, more often of 1 to 10 residues. Typical amino acid residues used for linking are tyrosine, cysteine, lysine, glutamic and aspartic acid, or the like.

Pharmaceutical Compositions

According to some embodiments, the present invention provides a pharmaceutical composition comprising as an active ingredient an agent capable of mediating AAT autophagic activity, and a pharmaceutically acceptable carrier, excipient or diluent.

As used herein, a “pharmaceutical composition” refers to a preparation of one or more of the active ingredients described herein, with other components such as physiologically suitable carriers and excipients. The purpose of a pharmaceutical composition is to facilitate administration of a compound to a subject.

Hereinafter, the phrases “therapeutically acceptable carrier” and “pharmaceutically acceptable carrier”, which may be used interchangeably, refer to a carrier or a diluent that does not cause significant irritation to an organism and does not abrogate the biological activity and properties of the administered compound.

Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols. Administration could be also via inhalation.

In another embodiment of the present invention, a therapeutic composition further comprises a pharmaceutically acceptable carrier. As used herein, a “carrier” refers to any substance suitable as a vehicle for delivering of the agents or molecule of the present invention to a suitable in vivo or in vitro site. As such, carriers can act as a pharmaceutically acceptable excipient of a therapeutic composition of the present invention. Carriers of the present invention include: (1) excipients or formularies that transport, but do not specifically target a molecule to a cell (referred to herein as non-targeting carriers); and (2) excipients or formularies that deliver a molecule to a specific site in a subject or a specific cell (i.e., targeting carriers). Examples of non-targeting carriers include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity.

Therapeutic compositions of the present invention can be sterilized by conventional methods.

Targeting carriers are herein referred to as “delivery vehicles”. Delivery vehicles of the present invention are capable of delivering a therapeutic composition of the present invention to a target site in a subject. A “target site” refers to a site in a subject to which one desires to deliver a therapeutic composition. Examples of delivery vehicles include, but are not limited to, artificial and natural lipid-containing delivery vehicles. Natural lipid-containing delivery vehicles include cells and cellular membranes. Artificial lipid-containing delivery vehicles include liposomes and micelles. A delivery vehicle of the present invention can be modified to target to a particular site in a subject, thereby targeting and making use of a nucleic acid molecule of the present invention at that site. Suitable modifications include manipulating the chemical formula of the lipid portion of the delivery vehicle and/or introducing into the vehicle a compound capable of specifically targeting a delivery vehicle to a preferred site, for example, a preferred cell type. Specifically targeting refers to causing a delivery vehicle to bind to a particular cell by the interaction of the compound in the vehicle to a molecule on the surface of the cell. Suitable targeting compounds include ligands capable of selectively (i.e., specifically) binding another molecule at a particular site. Examples of such ligands include antibodies, antigens, receptors and receptor ligands. For example, an antibody specific for an antigen found on the surface of a target cell can be introduced to the outer surface of a liposome delivery vehicle so as to target the delivery vehicle to the target cell. Manipulating the chemical formula of the lipid portion of the delivery vehicle can modulate the extracellular or intracellular targeting of the delivery vehicle. For example, a chemical can be added to the lipid formula of a liposome that alters the charge of the lipid bilayer of the liposome so that the liposome fuses with particular cells having particular charge characteristics.

According to some embodiments wherein the enhancement of autophagy is attempted, the pharmaceutical composition may further comprise an additional autophagic cell death. In an exemplary embodiment, the additional autophagic cell death is tamoxifen.

Therapeutic Use

According to some embodiments, the peptides or agents of the present invention are useful in regulating autophagic cell death by modulating AAT activity, including but not limited to reducing or enhancing AAT expression. Reducing AAT activity (e.g., inhibition or down regulation of intracellular AAT) is beneficial in the treatment of hyper proliferative diseases such as cancer. Promoting AAT activity is beneficial in the treatment of diseases characterized by excessive cell death such as neurodegenerative diseases.

The term “subject” includes humans, and non-human animals amenable to therapy, e.g., preferably mammals and animals susceptible to an autophagy associated disease, such as a disease associated with misfolded protein aggregates, including non-human primates, transgenic animals, mice, rats, dogs, cats, rabbits, pigs, chickens, sheep, horses, and cows. Preferably, the subject is a human subject.

“Treating” within the scope of the present invention comprises reducing, inhibiting, ameliorating or preventing symptoms or molecular events associated with the autophagy related pathology to be treated.

According to other embodiments, the present invention provides a method for treating a disease or disorder characterized by excessive cellular death in a subject in need thereof, comprising administering to the subject a therapeutically effective amount of a pharmaceutical composition comprising alpha-1 antitrypsin, and a pharmaceutically acceptable carrier.

According to some embodiments, the present invention is directed to a method for treating cancer in a subject in need thereof, comprising administering to the subject a pharmaceutical composition comprising a therapeutically effective amount of an agent capable of reducing AAT activity or expression.

In another embodiment, the methods of treating cancer include a method for inhibiting tumor progression in a subject in need thereof and/or a method for inducing tumor regression in a subject in need thereof.

The anti-cancer agents of the present invention are active against a wide range of cancers, including carcinomas, sarcomas, myelomas, leukemias, lymphomas and mixed type tumors. Particular categories of tumors amenable to treatment include lymphoproliferative disorders, breast cancer, ovarian cancer, prostate cancer, cervical cancer, endometrial cancer, bone cancer, liver cancer, stomach cancer, colon cancer, pancreatic cancer, cancer of the thyroid, head and neck cancer, cancer of the central nervous system, cancer of the peripheral nervous system, skin cancer, kidney cancer, as well as metastases of all the above. Particular types of tumors amenable to treatment include: hepatocellular carcinoma, hepatoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing's tumor, leimyosarcoma, rhabdotheliosarcoma, invasive ductal carcinoma, papillary adenocarcinoma, melanoma, squamous cell carcinoma, basal cell carcinoma, adenocarcinoma (well differentiated, moderately differentiated, poorly differentiated or undifferentiated), renal cell carcinoma, hypernephroma, hypernephroid adenocarcinoma, bile duct carcinoma, choriocarcinoma, seminoma, embryonal carcinoma, Wilms' tumor, testicular tumor, lung carcinoma including small cell, non-small and large cell lung carcinoma, bladder carcinoma, glioma, astrocyoma, medulloblastoma, craniopharyngioma, ependymoma, pinealoma, retinoblastoma, neuroblastoma, colon carcinoma, rectal carcinoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma, non-Hodgkin's lymphoma.

According to certain embodiments, the cancer to be treated is selected from the group consisting of prostate cancer, breast cancer, skin cancer, colon cancer, lung cancer, pancreatic cancer, lymphoma, myeloma, leukemia, head and neck cancer, kidney cancer, ovarian cancer, bone cancer, liver cancer or thyroid cancer.

For example, in some embodiments, the tumor may include pediatric solid tumors, e.g., Wilms' tumor, hepatoblastoma and embryonal rhabdomyosarcoma, wherein each possibility represents a separate embodiment of the present invention. In other embodiments, the tumor includes, but is not limited to, germ cell tumors and trophoblastic tumors (e.g. testicular germ cell tumors, immature teratoma of the ovary, sacrococcygeal tumors, choriocarcinoma and placental site trophoblastic tumors), wherein each possibility represents a separate embodiment of the present invention. According to additional embodiments, the tumor includes, but is not limited to, epithelial adult tumors (e.g. bladder carcinoma, hepatocellular carcinoma, ovarian carcinoma, cervical carcinoma, lung carcinoma, breast carcinoma, squamous cell carcinoma in head and neck, colon carcinoma, renal cell carcinoma and esophageal carcinoma), wherein each possibility represents a separate embodiment of the present invention. In yet further embodiments, the tumor includes, but is not limited to, neurogenic tumors (e.g. astrocytoma, ganglioblastoma and neuroblastoma), wherein each possibility represents a separate embodiment of the present invention. In another embodiment, the tumor is prostate cancer. In another embodiment, the tumor is pancreatic cancer. In other embodiments, the tumor includes, for example, Ewing sarcoma, congenital mesoblastic nephroma, gastric adenocarcinoma, parotid gland adenoid cystic carcinoma, duodenal adenocarcinoma, T-cell leukemia and lymphoma, nasopharyngeal angiofibroma, melanoma, osteosarcoma, uterus cancer and non-small cell lung carcinoma, wherein each possibility represents a separate embodiment of the present invention.

In other embodiments, the compositions and methods of the present invention may be used to treat any neurodegenerative disease. In certain embodiments, the neurodegenerative disease is a proteinopathy, or protein-folding disease. Examples of such proteinopathies include, but are not limited to, Alzheimer's disease, Parkinson's disease, Lewy Body Dementia, ALS, Huntington's disease, spinocerebellar ataxias and spinobulbar muscular atrophy. In other embodiments, the methods of the present invention can be used to treat any neurodegenerative disease. Neurodegenerative diseases treatable by the methods of the present invention include, but are not limited to, Adrenal Leukodystrophy, alcoholism, Alexander's disease, Alper's disease, Alzheimer's disease, Amyotrophic lateral sclerosis, ataxia telangiectasia, Batten disease, bovine spongiform encephalopathy, Canavan disease, cerebral palsy, cockayne syndrome, corticobasal degeneration, Creutzfeldt-Jakob disease, familial fatal insomnia, frontotemporal lobar degeneration, Huntington's disease, HIV-associated dementia, Kennedy's disease, Krabbe's disease, Lewy body dementia, neuroborreliosis, Machado-Joseph disease, multiple system atrophy, multiple sclerosis, narcolepsy, Niemann Pick disease, Parkinson's disease, Pelizaeus-Merzbacher disease, Pick's disease, primary lateral sclerosis, prion diseases, progressive supranuclear palsy, Refsum's disease, Sandhoff disease, Schilder's disease, subacute combined degeneration of spinal cord secondary to pernicious anaemia, Spielmeyer-Vogt-Sjogren-Batten disease, spinocerebellar ataxia, spinal muscular atrophy, Steele-Richardson-Olszewski disease, Tabes dorsalis and toxic encephalopathy. Each possibility represents a separate embodiment of the present invention.

“Protein aggregation” within the scope of the present invention includes the phenomenon of at least two polypeptides contacting each other in a manner that causes either one of the polypeptides to be in a state of de-solvation. This may also include a loss of the polypeptide's native function or activity.

“Protein-aggregation-associated disease” within the scope of the present invention includes any disease, disorder, and/or affliction, protein-aggregation-associated disease including neurodegenerative disorders and myodegenerative disorders.

A subject in need thereof may also include, for example, a subject who has been diagnosed with a neurodegenerative disease or a subject who has been treated for a neurodegenerative disease, including subjects that have been refractory to the previous treatment. A subject in need thereof may also include, for example, a subject who has been diagnosed with a proteinopathy, including subjects that have been refractory to previous treatment.

Having now generally described the invention, the same will be more readily understood through reference to the following examples, which are provided by way of illustration and are not intended to be limiting of the present invention.

### EXAMPLES

### Materials and Methods

Cell Culture and Treatments

The human breast cancer MCF-7 cell line was cultured in DMEM (Invitrogen, 41965) supplemented with 10% FBS (Beit Haemek, 04-121-1A), 300 mg/l L-glutamine (Beit Haemek, 03-020-1B), 100 U/ml penicillin, 100 μg/ml streptomycin and 12.5 U/ml nystatin (Beit Haemek, 03-032-1B) and 100 U/ml recombinant human insulin (Beit Haemek, 01-818-1H), at 37° C. in 5% CO₂.

HT-29 colorectal adenocarcinoma cells were cultured in RPMI 1640 (Invitrogene, 21875) supplemented with 10% FBS, 300 mg/l L-glutamine, 100 U/ml penicillin, 100 μg/ml streptomycin and 12.5 U/ml nystatin at 37° C. in 5% CO₂.

Cells were plated in 24-wells plate or 96-well plates at a concentration of 5·10⁴ cells/ml. A day later the cells were treated with different concentrations of tamoxifen or TPCK as indicated below.

Labeling of Autophagic Vacuoles with Monodansylcadaverine (MDC)

Following treatment, the cells were incubated with 0.05 mM MDC in PBS at 37° C. for 10 minutes (Biederbick et al., Eur J Cell Biol. 1995 January; 66(1):3-14). Intracellular MDC was measured by fluorescence photometry (excitation wavelength 380 nm, emission filter 525 nm) in a TECAN SPECTRA Fluor Plus microplate reader. The MDC fluorescence intensity was correlated to cell density as measured by XTT reduction assay performed in parallel treated samples. The results are expressed as fluorescence/cell.

For visualization of MDC-labeled vacuoles, cells were grown on coverslips, incubated with 0.05 mM MDC at 37° C. for 10 minutes. After incubation, cells were washed four times with PBS and immediately analyzed by fluorescence microscopy.

XTT Colorimetric Assay

XTT assay is used for the quantification of cell proliferation and viability, where XTT (tetrazolium salt) is metabolized by viable cells only to a water-soluble formazan dye that can be measured spectrophotometrically. Briefly, 100 μl of cell suspension (10⁵ cells/ml) were added to a 96-well plate, 10 μl of different concentrations of the drugs (Tamoxifen, TPCK, TLCK) were added to the wells, and cells were incubated for various time points in a humidified incubator at 37° C. and 5% CO₂. A mixture of 25 μl of XTT (1 mg/ml) and PMS (100 mM) dissolved in DMEM was added to each well, and the cells were further incubated for 1 hour at 37° C. Finally, the absorbance of the dye was measured spectrophotometrically at 450 and 650 nm served as a reference wavelength.

Protein Lysates

Normal or treated 10⁷ cells were collected, washed twice with ice-cold PBS and resuspended in 0.5 ml ice-cold lysing buffer (50 mM Tris-HCl pH 7.5, 0.1% NP-40, 1 mM DTT, 0.25M sucrose, 2 mM MgCl₂). The cells were broken by politron (4 cycles of 7 sec) and the cell debris pelleted by centrifugation in an ultracentrifuge at 120,000×g for 30 min, at 4° C. The supernatant was used immediately or stored at −70° C. Protein content of each sample was determined with BioRad reagent according to Bradford's method using bovine serum albumin (BSA) as standard.

Enzyme Activity Assays

Trypsin activity was determined according to Bergmeyer et. al. (Bergmeyer, H. U., Gawehn, K. and Grassl, M. (1974) in Methods of Enzymatic Analysis (Bergmeyer, H. U. ed.) Volume 1, 2nd ed., 516-517, Academic Press, New York, N.Y.). All assays were set up at a total volume of 1 ml by adding 65 μl trypsin at final amount of 1.625 μg, 0.23 mM Na-benzoyl-L-arginine ethyl ester (BAEE) as substrate in 63 mM sodium phosphate, in the presence or absence of 100 μM or 125 μM AAT with or without different concentration of TPCK. Trypsin activity was measured by the cleavage of the substrate BAEE by measuring absorbance at 253 nm.

siRNA Transfection

For long-term transfection MCF-7 cells were transfected with 0.02 μM control negative siRNA or AAT siRNA (Qiagene, 5103648862) using the HiPerFect Transfection reagent. When the cells become confluent (after 72 h), they were split and transfected again.

Interaction Assay Between AAT to TRFCK

2 μg/μl AAT was incubated in vitro with 50 μM TRFCK in 37° c. for 30 min. After the incubation time the sample was subjected to 10% SDS-PAGE before being transferred to polyvinylidene difluoride (PVDF) membranes. At the end of the transfer the membrane was analyzed under UV lamp to track TRFCK fluorescence. The sample was then exposed to primary antibody anti-AAT. The secondary antibody was an HRP-conjugated anti-goat IgG (Jackson ImmunoResearch, USA) The membrane was then visualized using an ECL+plus Western Blotting Detection System.

Western Blot Analysis

Equal amounts of protein samples (40 μg/lane) were subjected to 10% SDS-PAGE before being transferred to polyvinylidene difluoride (PVDF) membranes. The samples were then exposed to the following primary antibodies: goat anti-AAT and goat anti-actin (Santa Cruz Biotechnology, Santa Cruz, Calif.) polyclonal antibody. The secondary antibody was an HRP-conjugated anti-goat IgG (Jackson ImmunoResearch, USA). The membranes were blocked with 5% skin milk/Tris-buffered saline containing 0.1% Tween-20 (TBST) at room temperature for 1 h, after which the samples were incubated overnight at 4° C. with each of the above primary antibodies. The membranes were then washed three times with TBST, followed by 1 h incubation at room temperature with the secondary antibody. The membranes were then washed three times with TBST and were visualized using an ECL+plus Western Blotting Detection System (Amersham Pharmacia Biotech, Piscataway, N.Y.).

### Example 1

### TPCK Induces Autophagy in MCF-7 and HT-29 Cell Lines

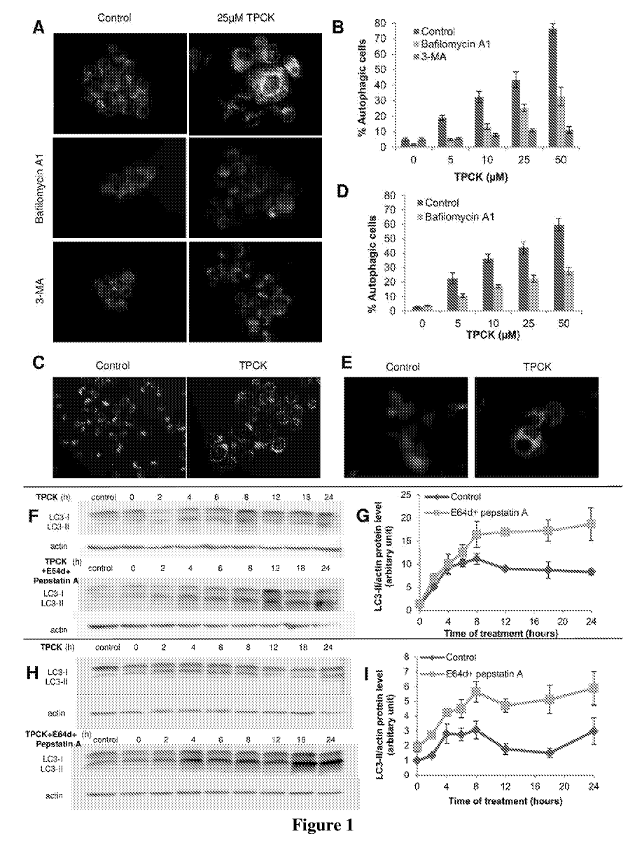

FIG. 1 shows that TPCK, a chymotrypsin-like protease inhibitor, induces autophagy in MCF-7 and HT-29 Cells. Autophagy was confirmed by the auto fluorescent marker of autolysosomes, monodensyl cadaverin (MDC). MDC staining of TPCK treated cells exhibited a dose dependent increase in fluorescence intensity and in the number and size of MDC-labeled vesicles (FIG. 1A-D). In addition, GFP-LC3 transfected MCF-7 cells treated with TPCK show accumulation of this label, indicating increased number of autophagosomes (FIG. 1E). Moreover, the autophagy inhibitors 3-Methyladenine (3-MA) (which blocks autophagosome formation via the inhibition of type III phosphatidylinositol 3-kinases (PI-3K) and bafilomycin A1 (a V-ATPase inhibitor which prevents maturation of autolysosomes) both suppressed MDC uptake in MCF-7 cells treated with TPCK (FIG. 1A, C, D) (mean±s.e. of at least four independent experiments). Furthermore, western blots of TPCK-treated cells revealed the appearance of LC3-II (FIG. 1F-I), one of the hallmarks of autophagy. In addition, co-incubation with the lysosomal protease inhibitors E64d and pepstatin A (which blocks the last steps of autophagosome fusion with the lysosome and prevent protein degradation) enhanced TPCK-induced accumulation of LC3-II, confirming that TPCK induces dynamic autophagy in MCF-7 and HT-29 cells.

### Example 2

### Alpha-1-Antitrypsin (AAT) Binds TPCK, a Chymotrypsin-Like Protease Inhibitor

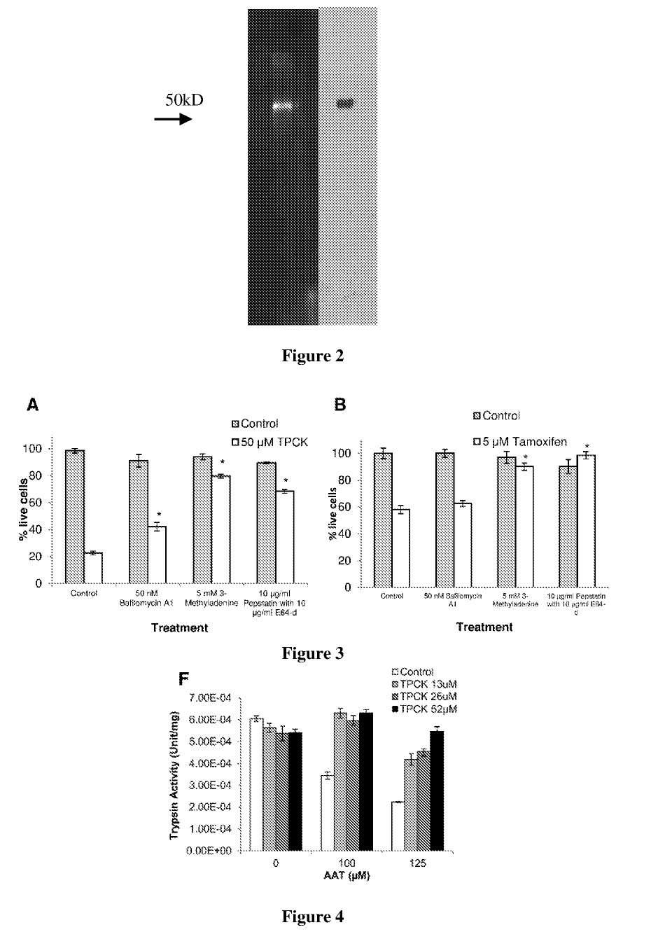

AAT was incubated in vitro with 50 μM TRFCK (a fluorescence analog of TPCK, N-tosyl-L-phenylalanine chloromethyl ketone) in 37° C. for 30 min. Following incubation, the sample was applied to SDS-PAGE electrophoresis and transferred to PVDF membrane. Thereafter, the membrane was analyzed under UV lamp to track TRFCK fluorescence (FIG. 2, track 1). In addition, Western blot analysis was performed using the same membrane with an antibody against AAT (FIG. 2, track 2). As seen in FIG. 2 TRFCK covalently binds AAT. Tamoxifen was not able to compete with TRFCK on binding to AAT suggesting that it does not bind to the same site on AAT.

### Example 3

### TPCK and Tamoxifen Induce Autophagic Cell Death

FIG. 3 shows that TPCK and tamoxifen induced cell death in MCF-7 cells, as revealed by trypan blue staining. Moreover, addition, of 50 nM bafilomycin A1, 5 mM 3-Methyladenine or a combination of 10 m/ml pepstatin with 10 m/ml E64-d reduced TPCK or tamoxifen-induced cell death (FIG. 3A, B) further supporting the notion that cell death was induced by autophagy.

### Example 4

### TPCK Reduced Inhibition of Trypsin Activity by AAT

FIG. 4 shows that TPCK prevents AAT inhibition of trypsin activity As seen, TPCK by itself did not inhibit the trypsin activity. AAT inhibits trypsin activity. When TPCK was added the inhibition by AAT was abolished (*P<0.001, compared with control, by Student t test). The results indicate that TPCK binding to AAT reduces AAT inhibition of trypsin.

### Example 5

### AAT Plays a Role in Autophagic Cell Death Inhibition

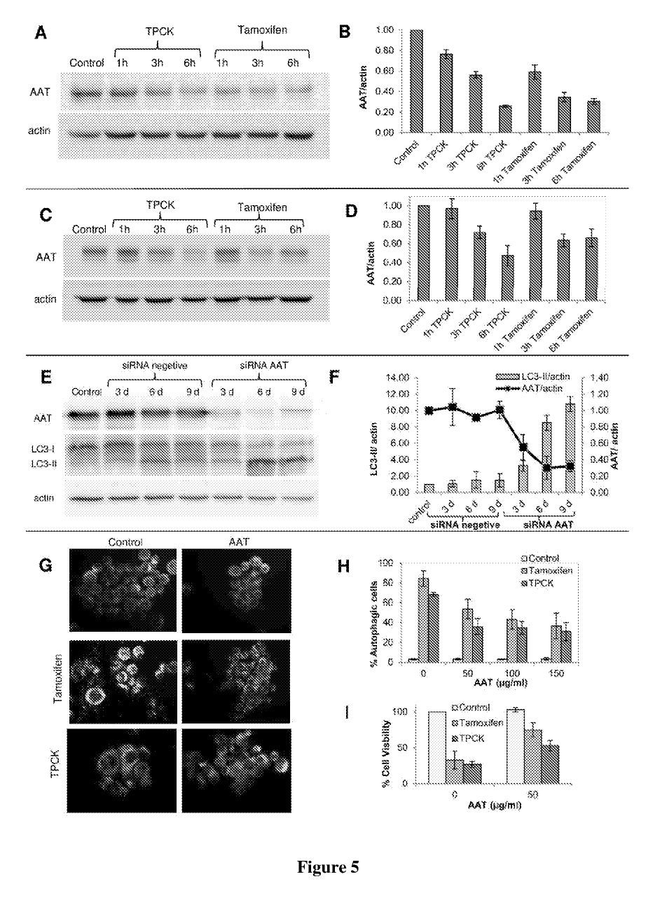

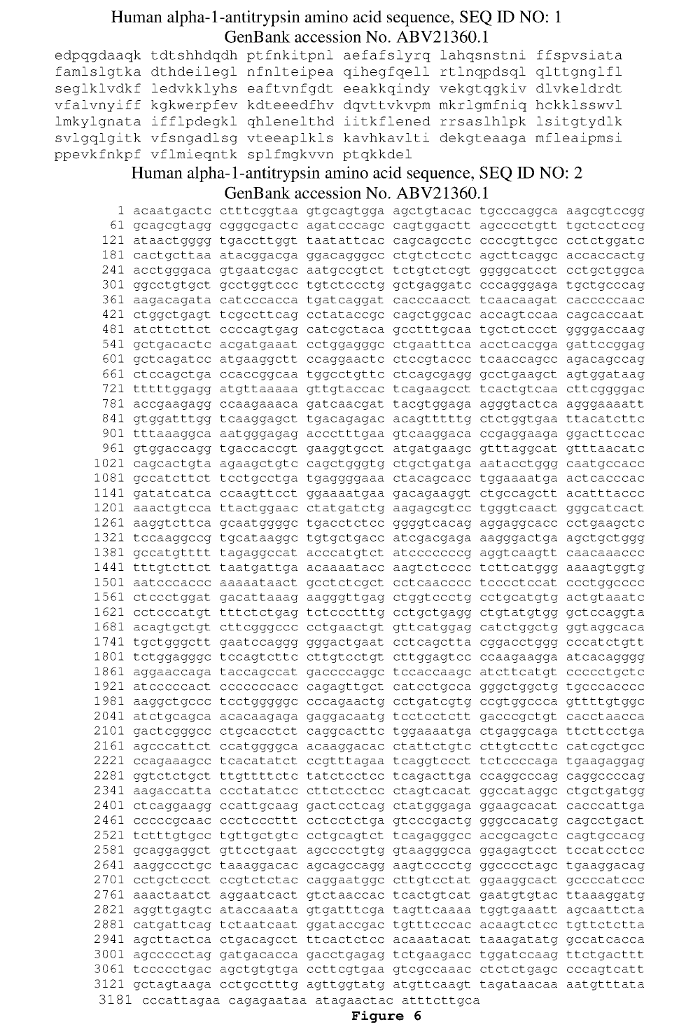

Western blot analysis of MCF-7 (FIG. 5A, B) and HT-29 (FIG. 5C, D) cell lysates reveals that AAT level decreased during TPCK and tamoxifen treatment as compared to actin. Since the half lifetime of AAT is relatively long, 3-4 days, the fast decrease in AAT levels following 1-3 hr treatment suggests that the protein undergoes degradation.

In addition, we further investigated whether inhibition of AAT in the cells will induce autophagy. MCF-7 cells transfected with siRNA for AAT showed increase in autophagy after 6 days as measured by Western blot for LC3-II (FIG. 5E-F).

Would externally added recombinant AAT to cells inhibit autophagic cell death? Addition of AAT to the cells prior to induction of autophagy by both TPCK and tamoxifen results in reduction in autophagy, assessed by MDC staining (FIG. 5G, H) and in cell death, determined by trypan blue staining (FIG. 5I). This suggests that recombinant AAT presumably competes with the native AAT on binding to TPCK and prevents autophagic cell death.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials and steps for carrying out various disclosed functions may take a variety of alternative forms without departing from the invention.

1. A method for treating cancer in a subject in need thereof, comprising administering to the subject a therapeutically effective amount of an agent that reduces alpha-1-antitrypsin activity or expression levels, thereby treating cancer in said subject.

2. The method of claim 1, wherein the alpha-1-antitrypsin is human alpha-1-antitrypsin.

3. The method of claim 1, wherein the human alpha-1-antitrypsin encoded by polynucleotide having a nucleic acid sequence as set forth in SEQ ID NO: 2.

4. The method of claim 1, wherein the agent is a hybridizing agent capable of hybridizing to nucleic acid encoding alpha-1-antitrypsin.

5. The method of claim 4, wherein the hybridizing agent comprises at least one nucleic acid sequence at least 85% complementary to a target sequence of about 12 to about 100 nucleotides of alpha-1-antitrypsin mRNA.

6. The method of claim 5, wherein the target sequence is from about 12 to about 50 nucleotides of alpha-1-antitrypsin mRNA.

7. The method of claim 6, wherein said target sequence is from about 12 to about 25 nucleotides of alpha-1-antitrypsin mRNA.

8. The method of claim 1, wherein the hybridizing agent is selected from a RNA interference (RNAi) molecule and an antisense molecule.

9. The method of claim 8, wherein the RNAi molecule is selected from a short interference RNA (siRNA), small hairpin RNA (shRNA) and microRNA (miRNA).

10. The method of claim 8, wherein the RNAi molecule comprises a polynucleotide having at least 90% identity to the target sequence of alpha-1-antitrypsin mRNA.

11. The method of claim 1, wherein the agent is an antisense molecule comprising a polynucleotide at least 90% complementary to a target sequence of alpha-1-antitrypsin.

12. The method of claim 1, wherein the agent is administered to said subject in the form of a pharmaceutical composition further comprising a pharmaceutically acceptable carrier, excipient or diluent.

13. The method of claim 1, wherein the cancer is a hematopoietic malignancy.

14. The method of claim 13, wherein the hematopoietic malignancy is selected from the group consisting of: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma and non-Hodgkin's lymphoma.

15. The method of claim 1, wherein the cancer is a solid malignancy.

16. The method of claim 15, wherein the solid malignancy is selected from the group consisting of: prostate cancer, breast cancer, skin cancer, colon cancer, lung cancer, pancreatic cancer, head and neck cancer, kidney cancer, ovarian cancer, cervix cancer, bone cancer, liver cancer, thyroid cancer and brain cancer.

17. The method according claim 1, wherein the subject is a mammal.

18. The method according to claim 17, wherein the subject is a human.

19. A method for treating a disease or disorder associated with excessive autophagy in a subject in need thereof, comprising administering to the subject a therapeutically effective amount of an active agent selected from (a) an isolated alpha-1-antitrypsin polypeptide comprising the amino acid sequence as set forth in SEQ ID NO: 1, or an active analog or fragment thereof; (b) an isolated nucleic acid molecule encoding alpha-1-antitrypsin polypeptide, the alpha-1-antitrypsin polypeptide comprises the amino acid sequence as set forth in SEQ ID NO: 1, or an active analog or fragment thereof; or (c) an expression vector comprising the isolated nucleic acid molecule of (b); thereby treating the disease or disorder associated with excessive autophagy in said subject.

20. The method of claim 19, wherein the nucleic acid molecule encoding alpha-1-antitrypsin has a nucleic acid sequence as set forth in SEQ ID NO: 1 or an active analog thereof.

21. The method of claim 19, wherein said alpha-1-antitrypsin is administered to said subject in the form of a pharmaceutical composition further comprising a pharmaceutically acceptable carrier, excipient or diluent.

22. The method of claim 19, wherein the disease or disorder associated with excessive autophagy is a neurodegenerative disease.

23. The method of claim 22, wherein the neurodegenerative disease is selected from the group consisting of: Alzheimer's disease, Huntington's disease, Parkinson's disease, neurodegeneration due to stroke, amyotrophic lateral sclerosis (ALS), prion disease, Pick's disease, Progressive Supranuclear Palsy (PSP), fronto-temporal dementia (FTD), pallido-ponto-nigral degeneration (PPND), Guam-ALS syndrome, pallido-nigro-luysian degeneration (PNLD) and cortico-basal degeneration (CBD).

24. The method of claim 19, wherein the disease or disorder is associated with cell death.

25. The method of claim 19, wherein the disease or disorder is associated with neuronal cell death.

26. The method of claim 19, wherein disease or disorder associated with excessive autophagy is pancreatitis.
